(19) United States
(12) Patent Application Publication
(10) Pub. No.: US 2016/0287379 A1
(43) Pub. Date: Oct. 6, 2016

(54) STABLE, PLATE-STYLE INTRAOCULAR LENS FACILITATING DECREASED INCISION SIZE

(71) Applicant: NOVARTIS AG, BASEL (CH)

(72) Inventors: ANTHONY McDOUGAL, COLLEGE STATION, TX (US); JAMES M. SCOTT, WEATHERFORD, TX (US); QUN PENG, ALISO VIEJO, CA (US)

(21) Appl. No.: 15/051,286

(22) Filed: Feb. 23, 2016

Related U.S. Application Data
(60) Provisional application No. 62/141,896, filed on Apr. 2, 2015.

(51) Int. Cl. A61F 2/16

(52) U.S. Cl. CPC A61F 2/16; A61F 2002/169053; A61F 2002/1689; A61F 2002/169

(57) ABSTRACT

An IOL includes an optic configured to be positioned along the optical axis of a patient's eye and a plurality of haptics extending from a periphery of the optic. Each of the plurality of haptics includes a proximal region extending from the periphery of the optic, an intermediate region extending from a distal end of the proximal region, and a distal region extending from a distal end of the intermediate region. The thickness of the distal region of each haptic is greater that the thickness of the intermediate region.

### FIELD

This present disclosure relates generally to cataract surgery and, more particularly, to a stable, plate-style intraocular lens facilitating decreased incision size.

### BACKGROUND

Visually impairing cataract, or clouding of the lens, is the leading cause of preventable blindness in the world. Presently, cataracts are treated by surgical removal of the affected lens and replacement with an artificial intraocular lens (“IOL”). FIG. 1 is a diagram of an eye 100 illustrating anatomical structures related to the surgical removal of a cataract and the implantation of an IOL. The eye 100 comprises an opacified lens 102, an optically clear cornea 104, and an iris 106. A lens capsule (capsular bag 108) located behind the iris 106 of the eye 100 contains the opacified lens 102. More particularly, the opacified lens 102 is seated between an anterior capsule segment (anterior capsule 110) and a posterior capsular segment (posterior capsule 112). The anterior capsule 110 and the posterior capsule 112 meet at an equatorial region 114 of the capsular bag 108. The eye 100 also comprises an anterior chamber 116 located in front of the iris 106 and a posterior chamber 118 located between the iris 106 and the capsular bag 108.

A common technique for cataract surgery is extracapsular cataract extraction (“ECCE”), which involves the creation of an incision near the outer edge of the cornea 104 and an opening in the anterior capsule 110 (i.e., an anterior capsulotomy) through which the opacified lens 102 is removed. The lens 102 can be removed by various known methods. One such method is phacoemulsification, in which ultrasonic energy is applied to the lens to break it into small pieces that are aspirated from the capsular bag 108. Thus, with the exception of the portion of the anterior capsule 110 that is removed in order to gain access to the lens 102, the capsular bag 108 may remain substantially intact throughout an ECCE. The intact posterior capsule 112 provides a support for the IOL and acts as a barrier to the vitreous humor within the posterior chamber 120 of the eye 100. Following removal of the opacified lens 102, an artificial IOL, which may be designed to mimic the transparency and refractive function of a healthy lens, is typically implanted within the capsular bag 108 through the opening in the anterior capsule 110.

To reduce trauma to the eye 100 resulting from ECCE, it may be desirable to minimize the size of the required incision in the cornea 104. As the incision size decreases, the volume of the IOL that can be inserted through the incision must also decrease. Reducing the volume of an IOL (e.g., by reducing the thickness of various portions of the IOL) may have adverse effects on the stability of the IOL once implanted in the capsular bag 108. Accordingly, a need exists for a stable IOL that facilitates decreased incision size.

Additionally, a frequent complication of ECCE is posterior capsule opacification (“PCO”), which results from the migration of residual lens epithelial cells from the equatorial region 114 of the capsular bag 108 toward the center of the posterior capsule 112. Subsequent to ECCE, the lens epithelial cells may proliferate between the IOL and the surface of the posterior capsule 112, leading to wrinkling and clouding of the normally clear posterior capsule 112. If clouding of the posterior lens capsule 112 occurs within the visual axis, then the patient will experience a decrease in visual acuity and may require additional surgery to correct the patient's vision. A widely utilized procedure to clear the visual axis of PCO is Neodymium: Yttrium-Aluminum-Garnet (“Nd/YAG”) laser capsulotomy, in which a laser beam is used to create an opening in the center of the cloudy posterior capsule 112. However, Nd/YAG laser capsulotomy exposes patients to the risk of severe complications that can lead to significant visual impairment or loss, such as retinal detachment, papillary block glaucoma, iris hemorrhage, uveitis/vitritis, and cystoid macula edema. Moreover, the laser energy is ordinarily directed though the IOL, which may damage the optics of the implant or disrupt its placement within the capsular bag 108. Accordingly, a need also exists for an IOL design that reduces or eliminates the occurrence of PCO (as opposed to merely treating PCO after implantation of the IOL).

### SUMMARY

The present disclosure generally concerns an IOL having a small volume facilitating insertion through an incision of reduced size but that retains stability after insertion. In certain embodiments, an IOL includes an optic configured to be positioned along the optical axis of a patient's eye and a plurality of haptics extending from a periphery of the optic. Each of the plurality of haptics includes a proximal region extending from the periphery of the optic, an intermediate region extending from a distal end of the proximal region, and a distal region extending from a distal end of the intermediate region. The thickness of the distal region of each haptic is greater that the thickness of the intermediate region.

Certain embodiments of the present disclosure may provide one or more technical advantages. As one exemplary advantage, the haptic design of the above-described IOL may provide increased haptic compression force as compared to certain conventional designs (e.g., open loop designs), which may provide increased IOL stability after insertion into the capsular bag of a patient's eye. As a result of the haptic compression force provided by the haptic design, the IOL may be constructed of materials having a lower modulus of elasticity without an adverse effect on post-insertion stability. Additionally, the cross sectional thickness of the IOL may be decreased without an adverse effect on post-insertion stability, thereby facilitating insertion via a smaller incision (e.g., a sub 2.0 mm incision). As another exemplary advantage, the plate-style design of the above-described IOL may reduce PCO as compared to certain conventional designs (e.g., open loop designs).

### BRIEF DESCRIPTION OF THE DRAWINGS

For a more complete understanding of the present disclosure and the advantages thereof, reference is now made to the following description taken in conjunction with the accompanying drawings in which like reference numerals indicate like features and wherein:

FIG. 1 is a diagram of an eye illustrating anatomical structures related to the surgical removal of a cataract and the implantation of an IOL;

FIGS. 2A-2B illustrate an exemplary plate-style intraocular lens, according to certain embodiments of the present disclosure;

FIG. 3 illustrates a cross-section of the plate-style intraocular lens depicted in FIGS. 2A-2B after insertion into the capsular bag of a patient's eye, according to certain embodiments of the present disclosure; and

FIGS. 4A-4C illustrates alternative exemplary configurations of the plate-style intraocular lens depicted in FIGS. 2A-2B, according to certain embodiments of the present disclosure.

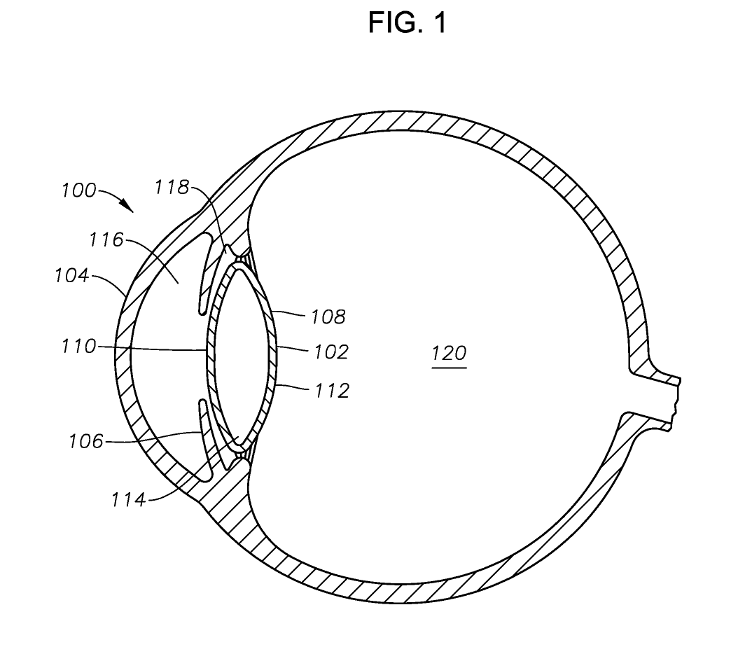

The skilled person in the art will understand that the drawings, described below, are for illustration purposes only. The drawings are not intended to limit the scope of the applicant's disclosure in any way.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described systems, devices, and methods, and any further application of the principles of the present disclosure are fully contemplated as would normally occur to one skilled in the art to which the disclosure relates. In particular, it is fully contemplated that the systems, devices, and/or methods described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

In general, the present disclosure relates to a plate-style intraocular lens having a small volume that facilitates insertion via a small incision (e.g., sub 2.0 mm) while maintaining stability once inserted into the capsular bag of a patient's eye. In certain embodiments, stability is maintained via a haptic design providing increased haptic force as compared to certain conventional haptic designs (e.g., open loop designs). More particularly, each haptic of the IOL of the present disclosure may include a distal region having a thickness greater than the remainder of the haptic. When implanted in the capsular bag 108 of a patient's eye, forces imparted on each haptic by the capsular bag 108 (e.g., due to contraction of the capsular bag 108 following surgery) may cause flexing in the haptic outside the distal region, resulting in increased haptic force. Accordingly, the haptic design described herein may (1) allow the IOL to be constructed of materials having a lower modulus of elasticity without adversely affecting post-insertion stability, and (2) allow the cross sectional thickness of the IOL to be decreased (thereby facilitating insertion via a smaller incision, such as a sub 2.0 mm incision) without adversely affecting post-insertion stability. Additionally, the plate-style design of the IOL described herein may reduce PCO as compared to certain conventional designs (e.g., open loop designs).

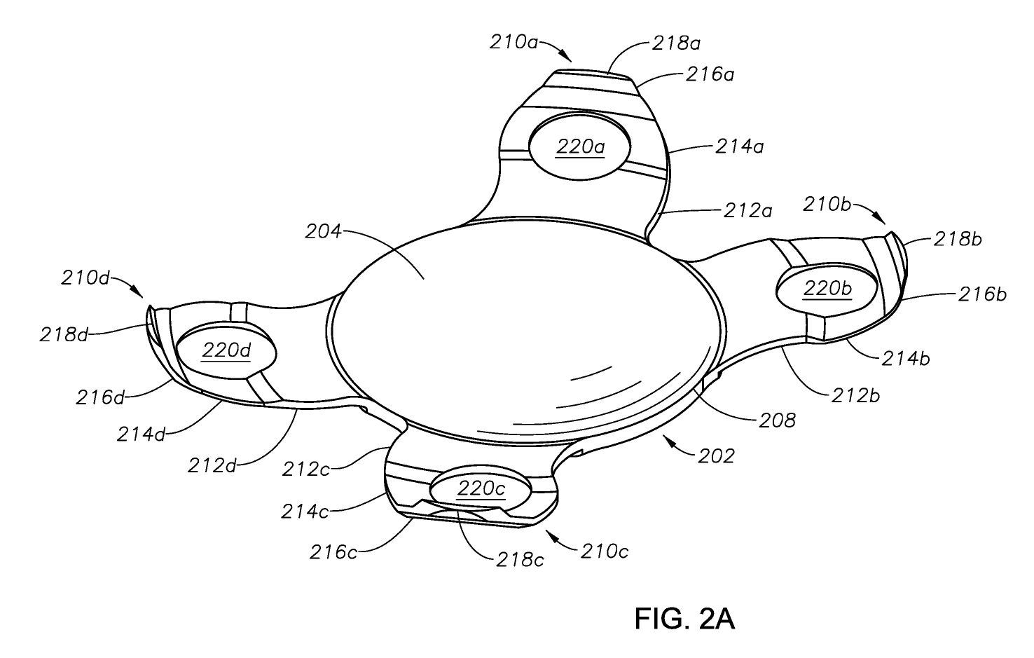

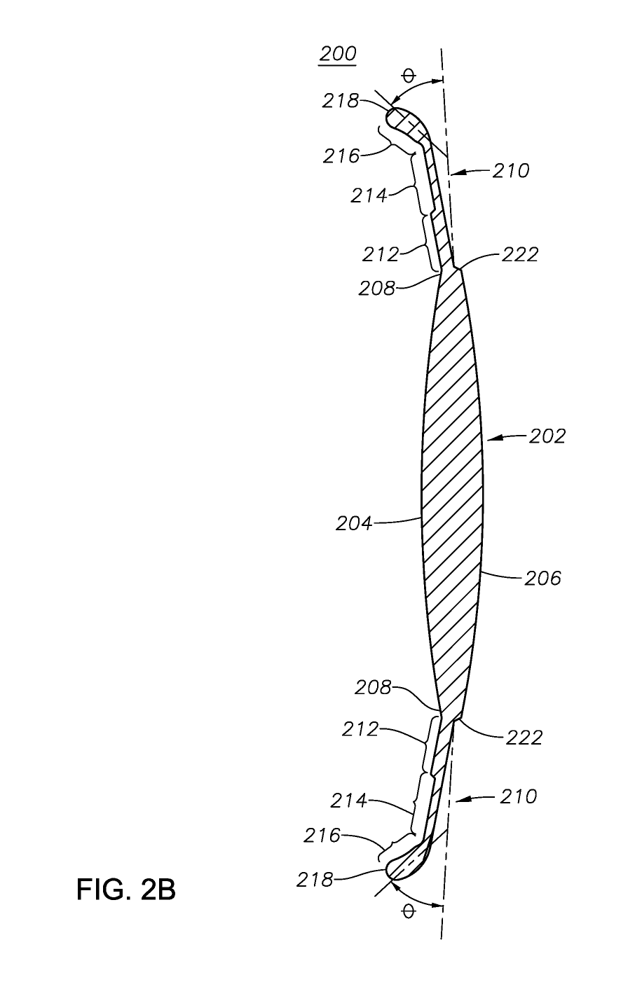

FIGS. 2A-2B illustrate an exemplary plate-style IOL 200, according to certain embodiments of the present disclosure. IOL 200 generally includes and optic 202 having an anterior surface 204, a posterior surface 206, and a periphery 208. Additionally, IOL 200 includes a plurality of haptics 210 extending outwardly from periphery 208. As used herein, the terms “anterior” and posterior” are intended to refer to directions when the IOL 200 is implanted in the eye (anterior being toward the cornea and posterior being toward the retina),

The present disclosure contemplates that IOL 200 may be any suitable device adapted to be inserted into a patient's eye to correct vision. IOL 200 may be a phakic IOL designed to be used in conjunction with the natural lens of a patient's eye to correct refractive errors such as myopia (near-sightedness), hyperopia (far-sightedness) astigmatism, coma or other higher order refractive errors (blurred vision due to poor light focusing on the retina due to an irregularly shaped cornea or, in some instances, an irregularly shaped natural lens). In such embodiments, IOL 200 may be inserted into either the anterior chamber 116 or posterior chamber 118, and may be supported by one or more of the sulcus and the iris 106. Alternatively, IOL 200 may be an aphakic or pseudophakic IOL that is inserted in the eye subsequent to removal of the natural lens due to disease (e.g., a cataract or clouding of the natural lens) and may restore, improve, or partially correct vision by providing a power comparable to that of the natural lens (as well as correcting myopia, hyperopia or other refractive errors). In such embodiments, IOL 200 may be inserted into the capsular bag 108 of the patient's eye after removal of the natural lens. Although the present disclosure contemplates that IOL 200 may be either phakic or aphakic, for purposed of simplicity it is assumed throughout the remainder this description that IOL 200 is an aphakic IOL configured for implantation into the capsular bag 108 of a patient's eye.

Optic 202 of IOL 200 may comprise any suitable optic for replacing a natural lens removed from a patient's eye and/or correcting errors in a patient's vision. For example, one or both of anterior surface 204 and posterior surface 206 may be curved such that the lens has an optical power corresponding to the patient's eye (such that light is properly focused on the retina). Additionally, optic 202 may have other features (e.g., spherical, aspheric, or toric features, as is known in the art) to further correct defects in the patient's vision.

IOL 200 may include a plurality of haptics 210 extending from the periphery 208 of optic 202. Haptics 210 may generally serve to contact a portion of the capsular bag 108 such that they collectively position and stabilize optic 202 in appropriate position along the optical axis of a patient's eye. Although IOL 200 is illustrated as including a particular number of haptics 210 (four haptics 210a-210d) having a particular shape, the present disclosure contemplates that IOL 200 may include any suitable number of haptics having any suitable shape, according to particular needs.

Each haptic 210 may include a proximal region 212 located nearest the periphery 208 of optic 202, an intermediate region 214 extending from the distal end of the proximal region, and a distal region 216 extending from the distal end of the intermediate region and terminating at a free end 218. Additionally, for each haptic 210, one or more of proximal region 212, intermediate region 214, and distal region 216 may have a thickness that differs from a thickness of other regions. For example, each haptic 210 may have a distal region 216 that has thickness greater than the intermediate region 214. The increased thickness of the distal region 216 may cause the haptics 210, in response to forces applied by the capsular bag 108 (e.g., resulting from the shrinking of the capsular bag 108 during the healing process) to bend in proximal region 212 and/or intermediate region 214 without substantial deformation in distal region 216. As a result, haptics 210 may maintain proper positioning of optic 202 when implanted in the capsular bag 108 while providing increased haptic force. The increased haptic force may allow for increased stability in the capsular bag (which may be particularly important when IOL 200 include a toric optic 202). Additionally, the increased haptic force may allow IOL 200 to be constructed of materials having a lower modulus of elasticity.

In certain embodiments, proximal region 212, intermediate region 214, and distal region 216 may each have different thicknesses. As just one example, IOL 200 may have an optic 202 having a maximum thickness of 0.680 mm, proximal region 212 may have a thickness of 0.111 mm, intermediate region may have a thickness of 0.177 mm, and distal region may have a thickness of 0.197 mm. In certain other embodiments, proximal region 212 and intermediate region 214 may each have the same thickness, which may be less than the thickness of distal region 216.

Although each region is described above as having a particular thickness, the present disclosure contemplates that the areas of transition between regions may have areas of varying thickness (where the thickness of the haptic 210 transitions from one region to the next). The particular thickness of each region described above may be a substantially constant thickness of the majority of each region (i.e., excluding the transition region) or an average thickness of each region. Additionally, although each of proximal region 212, intermediate region 214, and distal region 216 is depicted as covering a particular portion of each haptic 210, the present disclosure contemplates that each region may cover any suitable portion of each haptic 210.

In certain embodiments, haptics 210 may each include anteriorly-angled tip region spanning all or a portion of distal region 216, intermediate region 214, and/or proximal region 212. In the illustrated embodiment, anteriorly-angled tip region comprises the entirety of distal region 216; however, the present disclosure is not so limited.

The anteriorly-angled tip region of each haptic may be oriented at an angle 8 with respect to the plane of optic 202 when IOL 200 is in a unstressed state (i.e., when no force is applied to haptics 210 by capsular bag 108). In certain embodiments, 8 may be an angle in the range of 20 degrees to 70 degrees. As one particular example, θ may be an angle of approximately 45 degrees. After IOL 200 is inserted into the capsular bag 108 and the capsular bag 108 exerts a force on haptics 210 (e.g., due to contraction of the capsular bag during the healing process), the angle θ may increase due to flexing of the haptics 210 (e.g., in a region other than distal region 216, as described above) without displacing optic 202.

In certain embodiments, haptics 210 may each include an orifice 220 extending through the haptic 210. Each orifice 220 may be any suitable size and shape and may span one or more of proximal region 212, intermediate region 214, and distal region 216. For example, each orifice 220 may be substantially circular and sized so as to span a portion of proximal region 212 and intermediate region 214. In certain embodiments, orifices 220 may serve to reduce the overall volume of IOL 200, which may facilitate insertion through small corneal incision sizes (e.g., sub 2.0 mm incisions). Additionally, orifices 220 may facilitate circulation of aqueous humor located in anterior chamber 116 within the capsular bag, which may help to inhibit PCO.

In certain embodiments, IOL 200 may include an edge feature 222 extending around the periphery 208 of the posterior surface 206 of optic 202. Edge feature 222 may comprise a sharp transition between posterior surface 206 of optic 202 and haptics 210 or its periphery 208 (in areas where no haptic 210 is located). Edge feature 222 may comprise a feature having any suitable shape including a sharp transition (e.g., a square edge, a triangular lip, or any other suitable shape). As just one example, optic 202 may comprise a vaulted posterior surface that creates a sharp transition at periphery 208. When implanted in the capsular bag 108, edge feature 222 may contact a portion of the posterior capsule 112 and the sharp transition of edge feature 222 may inhibit migration of lens epithelial cells. As a result, edge feature 222 may inhibit PCO.

IOL 200 and components thereof may be constructed from one or more biocompatible materials. In particular, optic 202 may be constructed of a material that is optically transparent and smooth (e.g., an optical-quality surface). Exemplary materials include, hydrogels, silicones, acrylic materials, and other elastomeric polymers and soft plastics. For example, the silicone materials can be unsaturated terminated siloxanes, such as vinyl terminated siloxanes or multi-vinyl terminated siloxanes. Non-limiting examples include vinyl terminated diphenylsiloxane-dimethylsiloxane copolymers, vinyl terminated polyphenylmethylsiloxanes, vinyl terminated phenylmethylsiloxane-diphenyidimethylsiloxane copolymers, vinyl terminated polydimethylsiloxanes and methacrylate, and acrylate functional siloxanes. In other embodiments the lens-forming materials can be a hydrogel or a hydrophobic acrylic, such as the AcrySof® acrylic. Use of elastic/flexible materials can also enable the IOL 200 to be folded upon itself during implantation, thereby decreasing the size of the incision required to insert the IOL device 200 into the capsular bag 108.

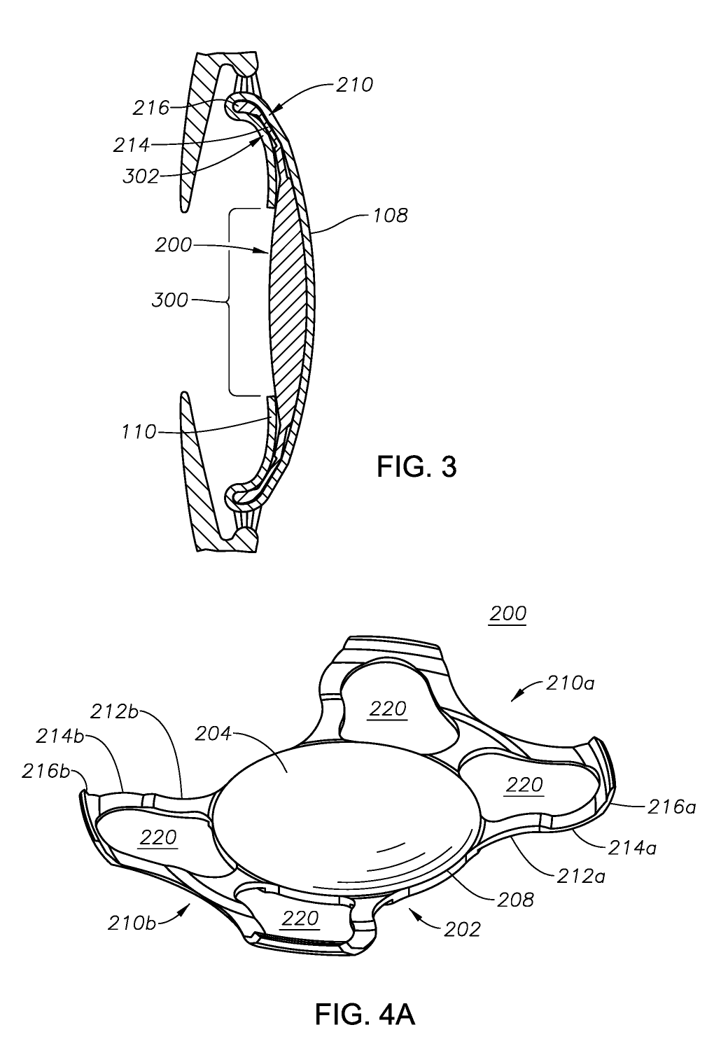

FIG. 3 illustrates a cross-section of the IOL 200 after insertion into the capsular bag 108 of a patient's eye, according to certain embodiments of the present disclosure. The capsular bag 108 is shown with the natural lens removed and an anterior capsulorhexis 300 (i.e., an area of the anterior capsule 110 that has been removed) through which IOL 200 is inserted into the capsular bag 108. As is illustrated, constriction of capsular bag 108 due to the healing process imparts a force of haptics 210, and that force cause haptics 210 to deflect in a region outside thickened distal region 216 (e.g., in region 302 of intermediate region 214).

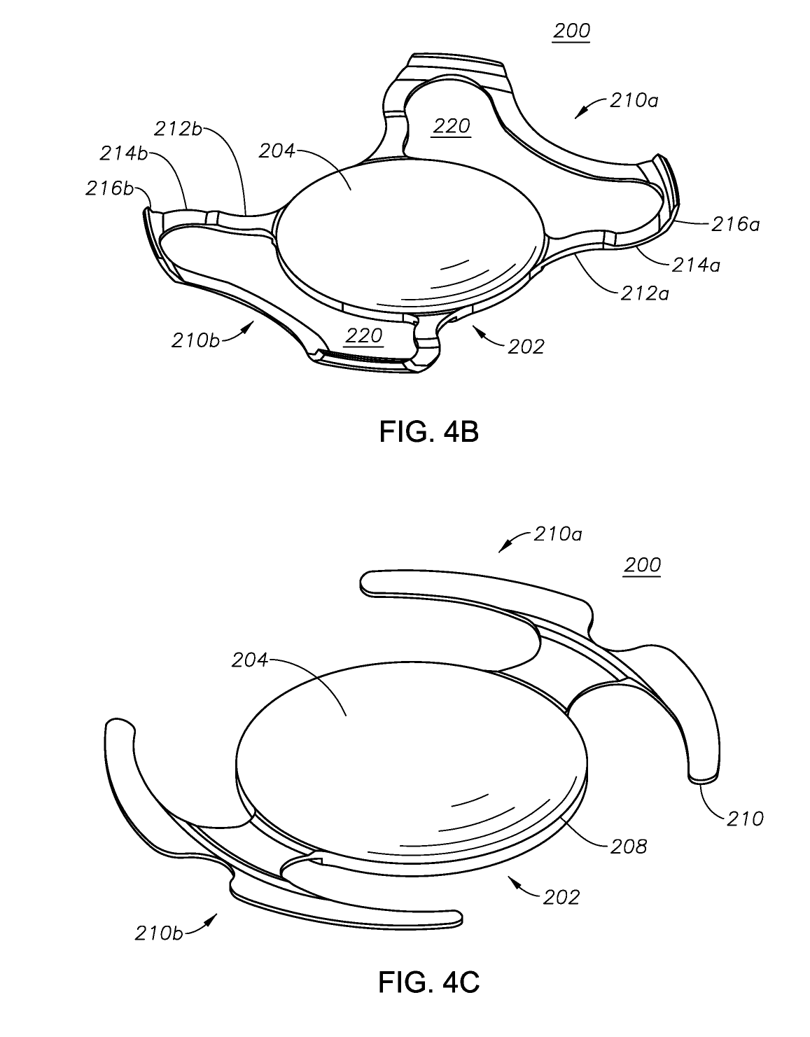

FIGS. 4A-4C illustrates alternative exemplary configurations of IOL 200, according to certain embodiments of the present disclosure. In particular, FIG. 3A illustrates an IOL 200 comprising two haptics 210a and 210b each comprising two irregularly shaped orifices 222. FIG. 3B illustrates a similar haptic design as FIG. 3A, except that each haptic 210 includes a single, larger orifice 222. Finally, FIG. 3C illustrates an IOL 200 comprising two haptics 210a and 210b without any orifices 222. Rather, distal region 216 is expanded radially to increase the contact area with the capsular bag 108. Although IOL 200 has been depicted as having particular haptic configurations for exemplary purposes, the present disclosure contemplates that IOL 200 may have any suitable number of additional haptic configurations consistent with the broad description included herein.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which alternatives, variations and improvements are also intended to be encompassed by the following claims.

1. An intraocular lens (IOL), comprising:
an optic configured to be positioned along the optical axis of a patient's eye;
a plurality of haptics extending from a periphery of the optic, each of the plurality of haptics comprising:
a proximal region extending from the periphery of the optic;
an intermediate region extending from a distal end of the proximal region; and
a distal region extending from a distal end of the intermediate region, the distal region having a thickness greater that a thickness of the intermediate region.

2. The IOL of claim 1, wherein each of the plurality of haptics comprises an anteriorly-angled tip region comprising at least a portion the distal region, the anteriorly-angled tip region being oriented at an angle relative to a plane of the optic.

3. The IOL of claim 1, wherein the angle is in the range of 20 to 70 degrees.

4. The IOL of claim 1, wherein each of the plurality of haptics are configured such that, when the IOL is positioned in a capsular bag of a patient's eye, a radial compressive force applied to the haptic by the capsular bag causes flexing to occur in the intermediate region while the distal region remains un-deformed.

5. The IOL of claim 1, wherein each of the plurality of haptics further comprises an orifice extending through the haptic.

6. The IOL of claim 5, wherein the orifice extends across at least a portion of the proximal region and at least a portion of the intermediate region.

7. The IOL of claim 1, wherein the thickness of the intermediate region is greater than a thickness of the proximal region.

8. The IOL of claim 1, wherein the thickness of the intermediate region is greater than a thickness of the proximal region.

9. The IOL of claim 1, further comprising an edge feature extending around the periphery of the optic, the edge feature comprising a sharp transition between a posterior surface of the optic and the periphery of the optic.

10. The IOL of claim 8, wherein the posterior surface of the optic is vaulted, creating the edge feature.

11. An intraocular lens (IOL), comprising:
an optic configured to be positioned along the optical axis of a patient's eye;
a plurality of haptics extending from a periphery of the optic, each of the plurality of haptics comprising:
a proximal region extending from the periphery of the optic;
an intermediate region extending from a distal end of the proximal region; and
a distal region extending from a distal end of the intermediate region, the distal region having a thickness greater that a thickness of the intermediate region; and
an anteriorly-angled tip region comprising at least a portion the distal region, the anteriorly-angled tip region being oriented at an angle relative to a plane of the optic
wherein each of the plurality of haptics are configured such that, when the IOL is positioned in a capsular bag of a patient's eye, a radial compressive force applied to the haptic by the capsular bag causes flexing to occur in the intermediate region while the distal region remains un-deformed.

12. The IOL of claim 11, wherein each of the plurality of haptics further comprises an orifice extending through the haptic.

13. The IOL of claim 12, wherein the orifice extends across at least a portion of the proximal region and at least a portion of the intermediate region.

14. The IOL of claim 11, wherein the thickness of the intermediate region is greater than a thickness of the proximal region.

15. The IOL of claim 11, wherein the thickness of the intermediate region is greater than a thickness of the proximal region.

16. The IOL of claim 11, further comprising an edge feature extending around the periphery of the optic, the edge feature comprising a sharp transition between a posterior surface of the optic and the periphery of the optic.

17. The IOL of claim 16, wherein the posterior surface of the optic is vaulted, creating the edge feature.
